(19) United States
(12) Patent Application Publication
(10) Pub. No.: US 2026/0227580 A1
(43) Pub. Date: Aug. 6, 2026

(54) Systems and Methods Including Procedural Barrier-Breaching Connectors and Connection-Establishing Devices

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 19/632,025

(22) Filed: Mar. 27, 2026

Related U.S. Application Data
(62) Division of application No. 17/512,501, filed on Oct. 27, 2021, now Pat. No. 12,596,232.
(60) Provisional application No. 63/106,809, filed on Oct. 28, 2020.

(51) Int. Cl. G02B 6/38; A61B 46/00

(52) U.S. Cl. CPC G02B 6/3817; A61B 46/00; G02B 6/3882

(57) ABSTRACT

Procedural barrier-breaching connectors and connection-establishing devices, and systems and methods thereof, for establishing portals in procedural barriers are disclosed. For example, a connection-establishing device can include a cap and an extension arm. The cap is configured to sit over a protruding portion of a first medical device with a procedural barrier between the connection-establishing device and the first medical device. The extension arm is configured to push a male connector of a second medical device through the procedural barrier and into a female connector of the first medical device with the extension arm, which establishes one or more functional connections across the procedural barrier between the second medical device and the first medical device. An exemplary method for the connection-establishing device includes a cap-placing step, an articulating step, and a functional connection-establishing step for establishing the one-or-more functional connections.

### PRIORITY

This application is a division of U.S. patent application Ser. No. 17/512,501, filed Oct. 27, 2021, now U.S. Pat. No. 12,596,232, which claims the benefit of priority to U.S. Provisional Application No. 63/106,809, filed Oct. 28, 2020, each of which is incorporated by reference in its entirety into this application.

### BACKGROUND

Procedural fields are typically established about patients with one or more procedural barriers before medical procedures. For example, sterile fields can be established over or around patients by covering the patients with sterile drapes. Oftentimes, the medical procedures require multiple-use medical devices that cannot be sterilized, which mandate placement of such medical devices under the sterile drapes; however, sterile single-use medical devices often need to be functionally connected to the multiple-use medical devices. Some existing solutions rely on arbitrarily breaching sterile drapes to make functional connections between sterile single-use medical devices and multiple-use medical devices. But arbitrarily breaching such sterile drapes risks contaminating the sterile fields carefully established about the patients. That, and establishing some functional connections such as optical connections between the sterile single-use medical devices and the multiple-use medical devices can be difficult, particularly through arbitrary, self-made breaches in the sterile drapes. What is needed are procedural barrier-breaching connectors and connection-establishing devices for establishing safe, routine, functional connections between sterile single-use medical devices and multiple-use medical devices.

Disclosed herein are procedural barrier-breaching connectors and connection-establishing devices, as well as systems and methods thereof, for establishing portals in procedural barriers.

### SUMMARY

Disclosed herein is a connection system including, in some embodiments, a male connector and a female connector. The male connector includes a base, external threads around the base, and opposing wings about a portion of the base. The base includes a longitudinal through hole having a terminal opening configured for holding therein a ferrule including an optical fiber. The wings are opposite the terminal opening. The wings are configured for turning the male connector by hand along a longitudinal axis thereof. The female connector includes a receptacle and internal threads around the receptacle. The receptacle includes a longitudinal through hole having an internal opening configured for holding therein another ferrule including another optical fiber. The internal threads around the receptacle are complementary with the external threads of the male connector. The male and female connectors are configured to establish an optical connection between the optical fiber of the male connector and the other optical fiber of the female connector when the optical fiber and the other optical fiber are respectively held in the male connector and the female connector.

In some embodiments, the external threads around the base of the male connector and the internal threads around the receptacle of the female connector are electrically conductive. The male and female connectors are configured to establish an electrical connection between a conductive wire of the male connector and another conductive wire of the female connector when the conductive wire and the other conductive wire are respectively held in the male connector and the female connector and connected to the internal threads and the external threads.

In some embodiments, the external threads around the base of the male connector include a cutting edge configured to cut through a procedural barrier when the male connector is placed against the procedural barrier and turned by hand along the longitudinal axis thereof.

In some embodiments, the base of the male connector and the receptacle of the female connector are complementarily tapered.

Also disclosed herein is a connection system including, in some embodiments, a male connector and a female connector. The male connector includes two or more prongs and an optical-fiber holder. The two-or-more prongs extend to a common terminal end of the male connector. The optical-fiber holder is along a longitudinal axis of the male connector between the two-or-more prongs and short of the common terminal end. The optical-fiber holder is configured for holding therein a ferrule including an optical fiber. The female connector includes two or more prong receptacles and an optical-fiber receptacle. The two-or-more prong receptacles correspond in number to the two-or-more prongs and extend to a common terminal end of the female connector. The optical-fiber receptacle is along a longitudinal axis of the female connector between the two-or-more prong receptacles and commensurate with the common terminal end of the female connector. The optical-fiber receptacle is configured for holding therein another ferrule including another optical fiber. The male and female connectors are configured to establish an optical connection between the optical fiber of the male connector and the other optical fiber of the female connector when the optical fiber and the other optical fiber are respectively held in the male connector and the female connector.

In some embodiments, the two-or-more prongs of the male connector and two or more contacts respectively disposed within the two-or-more prong receptacles of the female connector are electrically conductive. The male and female connectors are configured to establish an electrical connection between a conductive wire of the male connector and another conductive wire of the female connector when the conductive wire and the other conductive wire are respectively coupled to the two-or-more prongs in the male connector and the two-or-more contacts in female connector.

In some embodiments, each prong of the two-or-more prongs of the male connector include a cutting edge configured to cut through a procedural barrier when the male connector is placed against the procedural barrier and pushed by hand therethrough.

Also disclosed herein is a connection system including a male connector and a female connector. The male connector includes a sleeve, a cutting edge around a major opening of the sleeve, and a holed stopper. The sleeve has a through hole configured to allow a cable including an optical fiber to be slidably disposed therein. The cutting edge is opposite the through hole. The cutting edge is configured to cut through a procedural barrier when the sleeve is placed against the procedural barrier and turned by hand along a longitudinal axis of the sleeve. The holed stopper is configured to be disposed over the cable or a ferrule around the optical fiber. The holed stopper is configured to stop the cable from slipping though the through hole. The female connector includes an optical-fiber receptacle. The optical-fiber receptacle includes a longitudinal through hole having an internal opening configured for holding therein another ferrule including another optical fiber. The male and female connectors are configured to establish an optical connection between the optical fiber of the male connector and the other optical fiber of the female connector when the optical fiber and the other optical fiber are respectively held in the male connector and the female connector.

In some embodiments, the sleeve of the male connector has internal threads around an inside of the sleeve and the female connector has complementary external threads around an outside of the optical-fiber receptacle. The internal threads of the male connector and the external threads of the female connector are configured for screwing together the male connector and the female connector.

Also disclosed herein is a connection system including a male connector and a female connector. The male connector includes a head, a splittable shank extending from the head, and a spiral cutting edge around the splittable shank. The head has a through hole configured to allow a cable including an optical fiber to be slidably disposed therein. The splittable shank is configured to split along its length to allow a ferrule including the optical fiber of the cable to be pushed past a terminal end of the male connector. The spiral cutting edge is configured to cut through a procedural barrier when the male connector is placed against the procedural barrier and turned by hand along the longitudinal axis thereof. The female connector includes an optical-fiber receptacle. The optical-fiber receptacle includes a longitudinal through hole having an internal opening configured for holding therein another ferrule including another optical fiber. The male and female connectors are configured to establish an optical connection between the optical fiber of the male connector and the other optical fiber of the female connector when the optical fiber and the other optical fiber are respectively held in the male connector and the female connector.

In some embodiments, the splittable shank is configured to split in the optical-fiber receptacle to allow the ferrule including the optical fiber of the cable to be pushed past the terminal end of the male connector.

Also disclosed herein is a connection-establishing device including, in some embodiments, a cap and an extension arm. The cap is configured to sit over a protruding portion of a first medical device with a procedural barrier between the connection-establishing device and the first medical device. The extension arm is coupled by a hinge pin to the cap. The extension arm is configured to push a male connector of a second medical device through the procedural barrier and into a female connector of the first medical device by articulating the extension arm and establishing one or more functional connections across the procedural barrier between the second medical device on a clinician-facing side of the procedural barrier and the first medical device on a patient-facing side of the procedural barrier.

Also disclosed herein is a connection-establishing device including, in some embodiments, a cap, bail wire coupled to the cap, and an extension arm coupled to the cap. The cap is configured to sit over a protruding portion of a first medical device with a procedural barrier between the connection-establishing device and the first medical device. The bail wire is coupled to the cap by ends of the bail wire in bores of the cap. The bail wire is configured to secure the cap over the protruding portion of the first medical device by way of a hinge mechanism. The extension arm is coupled by a hinge pin to the cap. The extension arm is configured to push a male connector of a second medical device through the procedural barrier and into a female connector of the first medical device by articulating the extension arm. The extension arm is thusly configured to establish one or more functional connections across the procedural barrier between the second medical device on a clinician-facing side of the procedural barrier and the first medical device on a patient-facing side of the procedural barrier.

Also disclosed herein is a method of a connection system including a connector-placing step, a connector-turning step, and optical connection-establishing step. The connector-placing step includes placing a bottom portion of a base of a male connector into a top portion of a receptacle of a female connector with a procedural barrier therebetween. The connector-turning step includes turning the male connector by hand along a longitudinal axis thereof by way of opposing wings about a portion of the base opposite the bottom portion of the base while placing the male connector against the procedural drape. The connector-turning step also includes cutting through the procedural barrier with external threads around the base of the male connector having a cutting edge. The optical connection-establishing step includes establishing an optical connection between an optical fiber disposed in a terminal opening in the bottom portion of the male connector and another optical fiber disposed in an internal opening of the female connector respectively held in the male connector and the female connector.

In some embodiments, the method further includes an electrical connection-establishing step. The electrical connection-establishing step includes establishing an electrical connection between a conductive wire of the male connector and another conductive wire of the female connector. The conductive wire and the other conductive wire are respectively connected to the external threads of the male connector and complementary internal threads around the receptacle of the female connector.

Also disclosed herein is a method of a connection system including, in some embodiments, a connector-placing step, a connector-pushing step, and an optical connection-establishing step. The connector-placing step includes placing two or more prongs of a male connector against a procedural barrier. The connector-pushing step includes pushing the two-or-more prongs through the procedural barrier into two or more prong receptacles of a female connector corresponding in number to the two-or-more prongs of the male connector. The connector-pushing step also includes cutting through the procedural barrier with a cutting edge of each prong of the two-or-more prongs of the male connector. The optical connection-establishing step includes establishing an optical connection between an optical fiber disposed in an optical-fiber holder along a longitudinal axis of the male connector between the two-or-more prongs and another optical fiber disposed in an optical-fiber receptacle along a longitudinal axis of the female connector between the two-or-more prong receptacles.

In some embodiments, the method further includes an electrical connection-establishing step. The electrical connection-establishing step includes establishing an electrical connection between a conductive wire coupled to the two-or-more prongs of the male connector and another conductive wire coupled to two or more contacts respectively disposed within the two-or-more prong receptacles of the female connector.

Also disclosed herein is a method of a connection system including, in some embodiments, a connector-placing step, a connector-turning step, and a cable-sliding step. The connector-placing step includes placing a bottom portion of a sleeve of a male connector over a top portion of a receptacle of a female connector with a procedural barrier therebetween. The connector-turning step includes turning the male connector by hand along a longitudinal axis of the sleeve while placing the male connector against the procedural drape. The connector-turning step also includes cutting through the procedural barrier with a cutting edge around a major opening of the sleeve opposite a through hole. The cable-sliding step includes sliding a cable including an optical fiber through a through hole opposite the bottom portion of the sleeve of the male connector into the receptacle of the female connector. The cable-sliding step also includes establishing an optical connection between the optical fiber and another optical fiber disposed in the receptacle of the female connector.

In some embodiments, the sleeve of the male connector has internal threads around an inside of the sleeve and the female connector has complementary external threads around an outside of the optical-fiber receptacle for screwing together the male connector and the female connector subsequent to cutting the procedural drape in the connector-turning step.

Also disclosed herein is a method of a connection system including, in some embodiments, a connector-placing step, a connector-turning step, a connector-inserting step, and a cable-sliding step. The connector-placing step includes placing a spiral cutting edge of a splittable shank of a male connector over a longitudinal through hole of a receptacle of a female connector with a procedural barrier therebetween. The connector-turning step includes turning the male connector by hand along a longitudinal axis thereof while placing the male connector against the procedural drape. The connector-turning step also includes cutting through the procedural barrier with the cutting edge of the splittable shank. The connector-inserting step includes inserting the splittable shank into the longitudinal through hole of the receptacle of the female connector and splitting the splittable shank along its length. The cable-sliding step includes sliding a cable including an optical fiber through a through hole in a head of the male connector opposite the splittable shank into the receptacle of the female connector. The cable-sliding step also includes establishing an optical connection between the optical fiber and another optical fiber disposed in the receptacle of the female connector.

Also disclosed herein is a method of a connection-establishing device including, in some embodiments, a cap-placing step, an articulating step, and a functional connection-establishing step. The cap-placing step includes placing a cap over a protruding portion of a first medical device with a procedural barrier between the connection-establishing device and the first medical device. The articulating step includes articulating an extension arm coupled by a hinge pin to the cap to push a male connector of a second medical device through the procedural barrier and into a female connector of the first medical device. The functional connection-establishing step includes establishing one or more functional connections across the procedural barrier between the second medical device on a clinician-facing side of the procedural barrier and the first medical device on a patient-facing side of the procedural barrier.

Also disclosed herein is a method of a connection-establishing device including, in some embodiments, a cap-placing step, a securing step, an articulating step, and a functional connection-establishing step. The cap-placing step includes placing a cap over a protruding portion of a first medical device with a procedural barrier between the connection-establishing device and the first medical device. The securing step includes securing the cap over the protruding portion of the first medical device by way of a hinge mechanism of a bail wire coupled to the cap by ends of the bail wire in bores of the cap. The articulating step includes articulating an extension arm coupled by a hinge pin to the cap to push a male connector of a second medical device through the procedural barrier and into a female connector of the first medical device. The functional connection-establishing step includes establishing one or more functional connections across the procedural barrier between the second medical device on a clinician-facing side of the procedural barrier and the first medical device on a patient-facing side of the procedural barrier.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates functionally connected single-use and multiple-use medical devices of an optical shape-sensing system in accordance with some embodiments.

FIG. 2 illustrates a first connection system in accordance with some embodiments.

FIG. 3 illustrates a first view of a second connection system in accordance with some embodiments.

FIG. 4 illustrates a second view of the second connection system in accordance with some embodiments.

FIG. 5 illustrates a first view of a third connection system in accordance with some embodiments.

FIG. 6 illustrates a second view of the third connection system in accordance with some embodiments.

FIG. 7 illustrates a male connector of the third connection system in accordance with some embodiments.

FIG. 8 illustrates a breach in a procedural barrier when breached by the male connector of FIG. 5 in accordance with some embodiments.

FIG. 9 illustrates a first view of a fourth connection system in accordance with some embodiments.

FIG. 10 illustrates a second view of the fourth connection system in accordance with some embodiments.

FIG. 11 illustrates a male connector of the fourth connection system in accordance with some embodiments.

FIG. 12 illustrates a first connection-establishing device in accordance with some embodiments.

FIG. 13 illustrates a second connection-establishing device in accordance with some embodiments.

FIG. 14 illustrates a third connection-establishing device in accordance with some embodiments.

FIG. 15 illustrates a fourth connection-establishing device in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

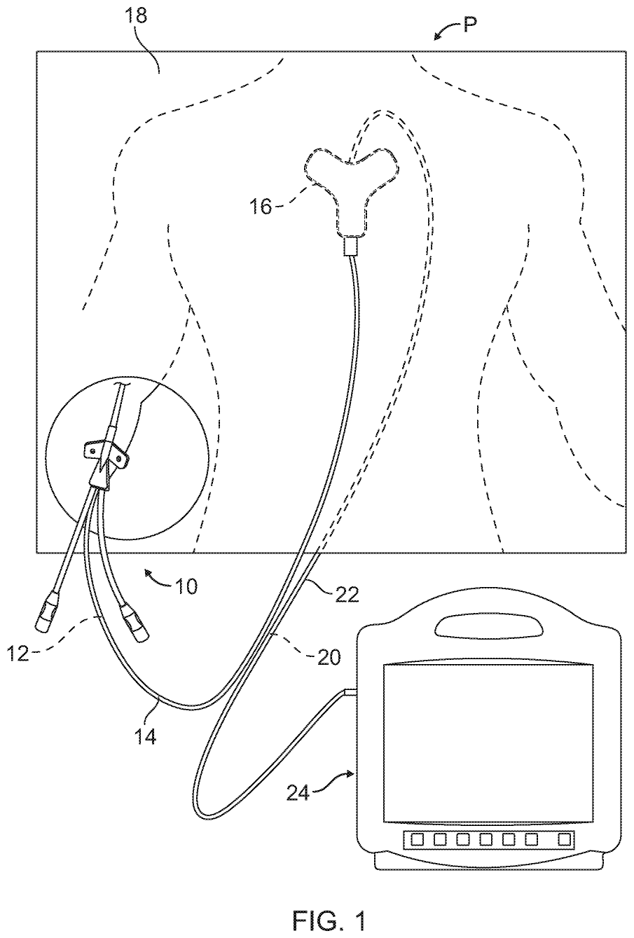

FIG. 1 illustrates functionally connected single-use and multiple-use medical devices of an optical shape-sensing system in accordance with some embodiments.

As set forth above, medical procedures often require multiple-use medical devices that cannot be sterilized, which mandate placement of such medical devices under sterile drapes; however, sterile single-use medical devices often need to be functionally connected to the multiple-use medical devices. Indeed, as shown, the optical shape-sensing system of FIG. 1 includes a single-use peripherally inserted central catheter (“PICC”) 10 having an optical-fiber stylet 12 disposed in an extension tube 14 functionally connected to a relay module 16 over a patient P but under a sterile drape 18 outside of a sterile field. The relay module 16, in turn, includes an optical fiber 20 disposed in a patch cable 22 functionally connected to a console 24 or an optical interrogator thereof that is also outside of the sterile field. Again, some existing solutions rely on arbitrarily breaching sterile drapes (e.g., the sterile drape 18) to make functional connections between sterile single-use medical devices (e.g., the PICC 10) and multiple-use medical devices (e.g., the relay module 16, the console 24, etc.). But arbitrarily breaching such sterile drapes risks contaminating the sterile fields carefully established about the patients. That, and establishing some functional connections such as optical connections between the sterile single-use medical devices and the multiple-use medical devices can be difficult, particularly through arbitrary, self-made breaches in the sterile drapes. What is needed are procedural barrier-breaching connectors and connection-establishing devices for establishing safe, routine, functional connections between sterile single-use medical devices and multiple-use medical devices.

Disclosed herein are procedural barrier-breaching connectors and connection-establishing devices, as well as systems and methods thereof, for establishing portals in procedural barriers.

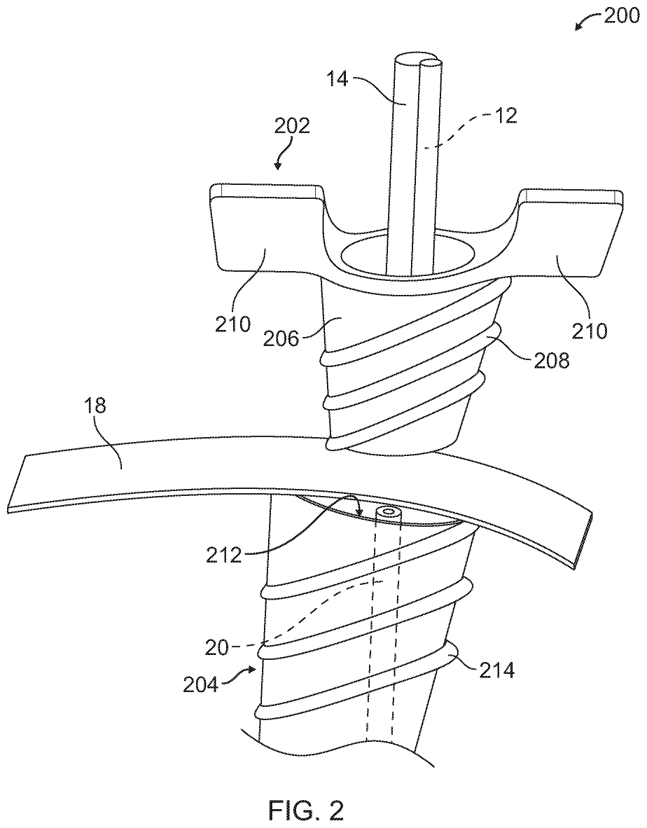

FIG. 2 illustrates a first connection system 200 in accordance with some embodiments.

As shown the connection system 200 includes a male connector 202 and a female connector 204.

The male connector includes a base 206, external threads 208 around the base 206, and opposing wings 210 about a portion of the base 206. The base 206 includes a longitudinal through hole having a terminal opening configured for holding therein a ferrule including an optical fiber such as the optical fiber of the optical-fiber stylet 12 as shown. The external threads 208 around the base 206 of the male connector 202 include a cutting edge configured to cut through a procedural barrier such as the sterile drape 18 when the male connector 202 is held against the procedural barrier and turned by hand along a longitudinal axis thereof, for example, when pressed into the receptacle 212 of the female connector 204 with the procedural barrier therebetween. The wings 210 are opposite the terminal opening. The wings 210 are configured for turning the male connector 202 by hand along the longitudinal axis thereof.

The female connector 204 includes a receptacle 212 and internal threads 214 around the receptacle 212. The receptacle 212 includes a longitudinal through hole having an internal opening (e.g., into the relay 16) configured for holding therein another ferrule including another optical fiber such as the optical fiber 20. The receptacle 212 of the female connector 204 and the base 206 of the male connector 202 are complementarily tapered. In addition, the internal threads 214 around the receptacle 212 are complementary with the external threads 208 of the male connector 202.

The male and female connectors 202 and 204 are configured to establish an optical connection between the optical fiber of the male connector 202 and the other optical fiber of the female connector 204 when the optical fiber and the other optical fiber are respectively held in the male connector 202 and the female connector 204.

The external threads 208 around the base 206 of the male connector 202 and the internal threads 214 around the receptacle 212 of the female connector 204 are electrically conductive. The male and female connectors 202 and 204 are configured to establish an electrical connection between a conductive wire of the male connector 202 and another conductive wire of the female connector 204 when the conductive wire and the other conductive wire are respectively held in the male connector 202 and the female connector 204 and connected to the external threads 208 and the internal threads 214.

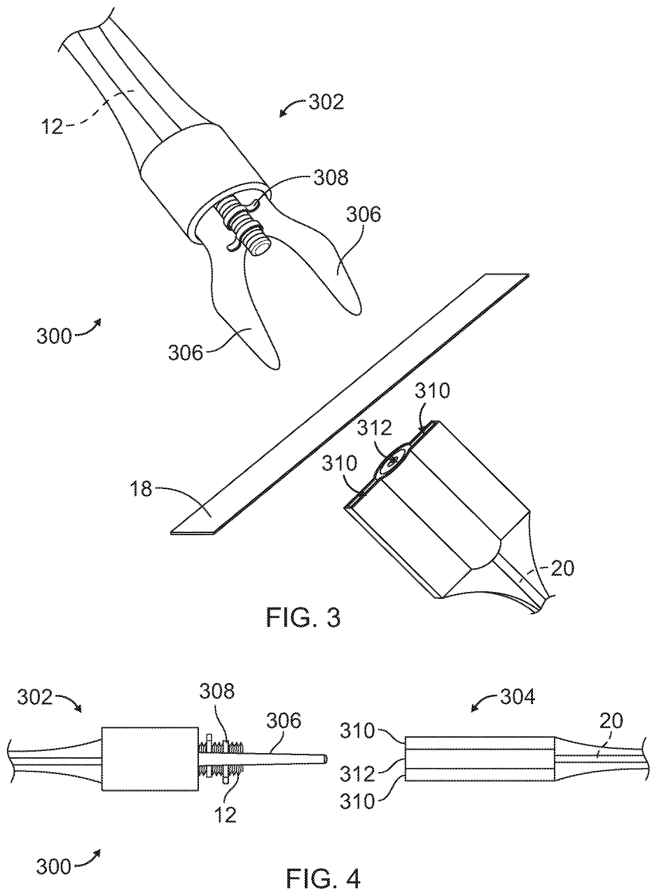

FIGS. 3 and 4 illustrate different views of a second connection system 300 in accordance with some embodiments.

As shown, the connection system 300 includes a male connector 302 and a female connector 304.

The male connector 302 includes two or more prongs 306 and an optical-fiber holder 308. The two-or-more prongs 306 extend to a common terminal end of the male connector 302. Each prong of the two-or-more prongs 306 of the male connector 302 includes a cutting edge configured to cut through a procedural barrier such as the sterile drape 18 when the male connector 302 is held against the procedural barrier and pushed by hand therethrough, for example, when pressed into the two-or-more prong receptacles 310 of the female connector 304 with the procedural barrier therebetween. The optical-fiber holder 308 is along a longitudinal axis of the male connector 302 between the two-or-more prongs 306 and short of the common terminal end. The optical-fiber holder 306 is configured for holding therein a ferrule including an optical fiber such as the optical fiber of the optical-fiber stylet 12 as shown.

The female connector 302 includes two or more prong receptacles 310 and an optical-fiber receptacle 312. The two-or-more prong receptacles 310 correspond in number to the two-or-more prongs 306 and extend to a common terminal end of the female connector 304. The optical-fiber receptacle 312 is along a longitudinal axis of the female connector 304 between the two-or-more prong receptacles 310 and commensurate with the common terminal end of the female connector 304. The optical-fiber receptacle 312 is configured for holding therein another ferrule including another optical fiber such as the optical fiber 20.

The male and female connectors 302 and 304 are configured to establish an optical connection between the optical fiber of the male connector 302 and the other optical fiber of the female connector 304 when the optical fiber and the other optical fiber are respectively held in the male connector 302 and the female connector 304.

The two-or-more prongs 306 of the male connector 302 and two or more contacts respectively disposed within the two-or-more prong receptacles 310 of the female connector 304 are electrically conductive. The male and female connectors 302 and 304 are configured to establish an electrical connection between a conductive wire of the male connector 302 and another conductive wire of the female connector 304 when the conductive wire and the other conductive wire are respectively coupled to the two-or-more prongs 306 in the male connector 302 and the two-or-more contacts in female connector 304.

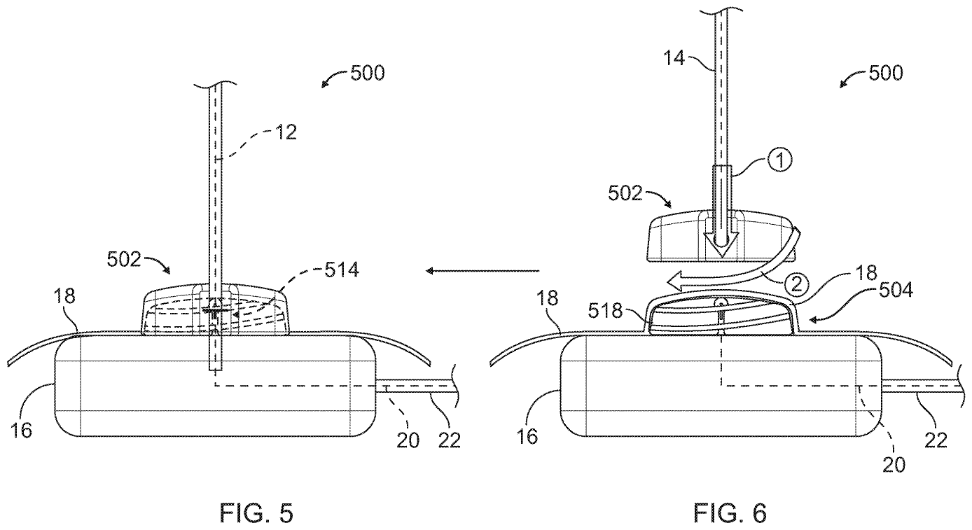

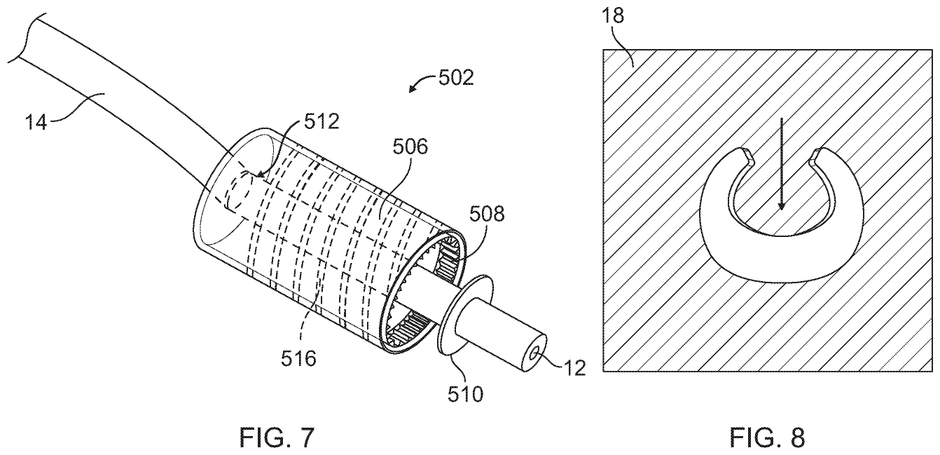

FIGS. 5 and 6 illustrate different views of the third connection system 500 in accordance with some embodiments. FIG. 7 illustrates a male connector 502 of the third connection system 500 in accordance with some embodiments. FIG. 8 illustrates a breach in a procedural barrier such as the sterile barrier 18 when breached by the male connector 502 of FIG. 7 in accordance with some embodiments. It should be understood that while the male connector 502 of FIGS. 5 and 6 appears different than that of FIG. 7, the male connector 502 can be in any form complementary to the female connector 504 set forth below.

As shown, the connection system 500 includes the male connector 502 and a female connector 504.

The male connector 502 includes a sleeve 506, a cutting edge 508 around a major opening of the sleeve 506, and a holed stopper 510 (e.g., an ‘O’-ring). The sleeve 506 has a through hole 512 configured to allow a cable (e.g., the extension tube 14) including an optical fiber (e.g., the optical fiber of the optical-fiber stylet 12) to be slidably disposed therein for sliding the cable into the sleeve 506 for cutting through a procedural barrier (e.g., the sterile drape 18) or out of the major opening at a terminal end of the male connector 502 and through a hole cut in the procedural drape (see FIG. 8) for making at least an optical connection. The cutting edge 508 is opposite the through hole 512. The cutting edge 508 is configured to cut through the procedural barrier when the sleeve 506 is held against the procedural barrier and turned by hand along a longitudinal axis of the sleeve 506. Notably, the through hole 512 of the sleeve 506 is also configured to allow the cable to be rotatably disposed therein for turning the sleeve 506 and cutting through the procedural barrier with the cutting edge 508 without twisting the cable. The holed stopper 510 is configured to be disposed over the cable or a ferrule around the optical fiber to stop the cable from slipping though the through hole when sliding the cable into the sleeve 506 for cutting through a procedural barrier.

The female connector 504 includes an optical-fiber receptacle 514. The optical-fiber receptacle 514 includes a longitudinal through hole having an internal opening configured for holding therein another ferrule including another optical fiber such as the optical fiber 20.

The male and female connectors 502 and 504 are configured to establish an optical connection between the optical fiber of the male connector 502 and the other optical fiber of the female connector 504 when the optical fiber and the other optical fiber are respectively held in the male connector 502 and the female connector 504.

Optionally, the male connector 502 and the female connector 504 can include complementary threads. Indeed, the sleeve 506 of the male connector 502 can have internal threads 516 around an inside of the sleeve 506 and the female connector 504 can have complementary external threads 518 around an outside of the optical-fiber receptacle 514. The internal threads 516 of the male connector 502 and the external threads 518 of the female connector 504 are configured for screwing together the male connector 502 and the female connector 504.

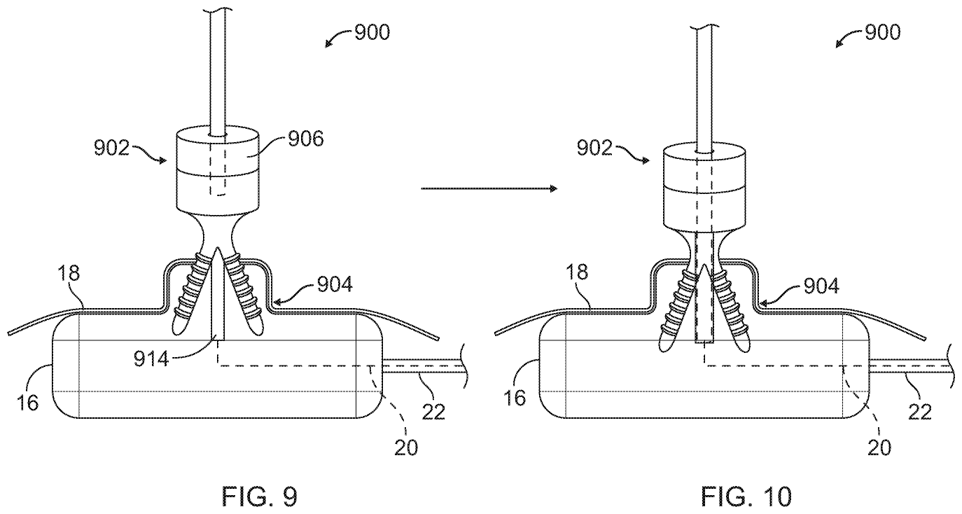

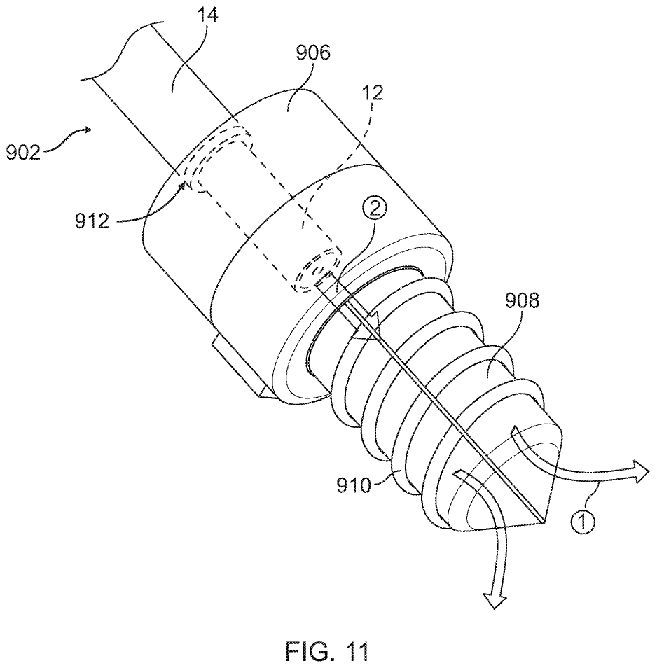

FIGS. 9 and 10 illustrate different views of a fourth connection system 900 in accordance with some embodiments. FIG. 9 illustrates a male connector 902 of the fourth connection system 900 in accordance with some embodiments.

As shown, the connection system 900 includes the male connector 902 and a female connector 904.

The male connector 902 includes a head 906, a splittable shank 908 extending from the head 906, and a spiral cutting edge 910 around the splittable shank 908. The head 906 has a through hole 912 configured to allow a cable (e.g., the extension tube 14) including an optical fiber (e.g., the optical fiber of the optical-fiber stylet 12) to be slidably disposed therein for sliding the cable out of the splittable shank 908 for making at least an optical connection. The splittable shank 908 is configured to split along its length to allow a ferrule including the optical fiber of the cable to be pushed past a terminal end of the male connector 902. The spiral cutting edge 910 is configured to cut through a procedural barrier (e.g., the sterile drape 18) when the male connector 902 is held against the procedural barrier and turned by hand along the longitudinal axis thereof.

The female connector 904 includes an optical-fiber receptacle 914. The optical-fiber receptacle 914 includes a longitudinal through hole having an internal opening configured for holding therein another ferrule including another optical fiber (e.g., the optical fiber 20). The optical-fiber receptacle 914 is configured to split the splittable shank 908 when the splittable shank 908 is inserted therein, which, in turn, allows the ferrule including the optical fiber of the cable to be pushed past the terminal end of the male connector 902 to establish at least an optical connection with the female connector 904. Indeed, the male and female connectors 902 and 904 are configured to establish an optical connection between the optical fiber of the male connector 902 and the other optical fiber of the female connector 904 when the optical fiber and the other optical fiber are respectively held in the male connector 902 and the female connector 904.

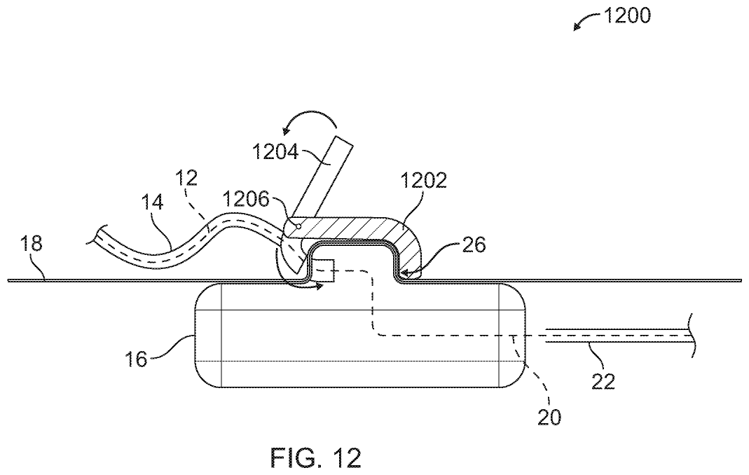

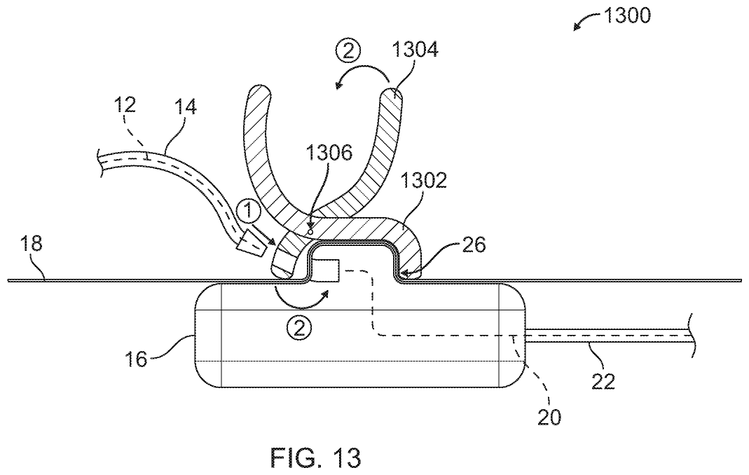

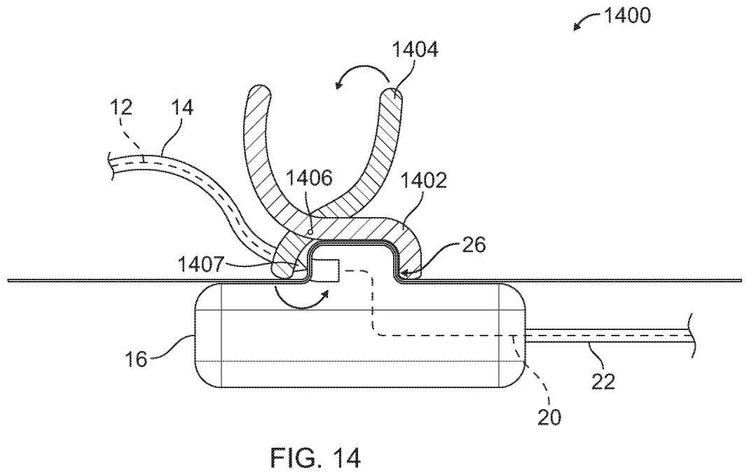

FIGS. 12, 13, and 14 respectively illustrate a first connection-establishing device 1200, a second connection-establishing device 1300, and a third connection-establishing device 1400 in accordance with some embodiments.

As shown, the connection-establishing device 1200, 1300, or 1400 includes a cap 1202, 1302, or 1402 and an extension arm 1204, 1304, or 1404. The cap 1202, 1302, or 1402 is configured to sit over a protruding portion 26 of, for example, a multiple-use medical device (e.g., the relay module 16) with a procedural barrier (e.g., the sterile drape 18) between the connection-establishing device 1200, 1300, or 1400 and the multiple-use medical device. The extension arm 1204, 1304, or 1404 is coupled by a hinge pin 1206, 1306, or 1406 to the cap 1202, 1302, or 1402. The extension arm 1204 or 1304 is configured to push a male connector (e.g., the male connector 202 or 302) of a single-use medical device (e.g., the PICC 10) through the procedural barrier and into a female connector (e.g., the female connector 204 or 304) of the multiple-use medical device, whereas the extension arm 1404 is configured to push a punch 1407 including an alternative male connector therein through the procedural barrier and into an alternative female connector. Regardless, by articulating the extension arm extension arm 1204, 1304, or 1404, one or more functional connections (e.g., an optical connection, an electrical connection, or an optical connection and an electrical connection) are established across the procedural barrier between the single-use medical device on a clinician-facing side of the procedural barrier and the multiple-use medical device on a patient-facing side of the procedural barrier.

Advantageously, the connection-establishing device 1200, 1300, or 1400 utilizes a mechanical advantage to decrease the force required to make the one-or-more functional connections through the procedural barrier.

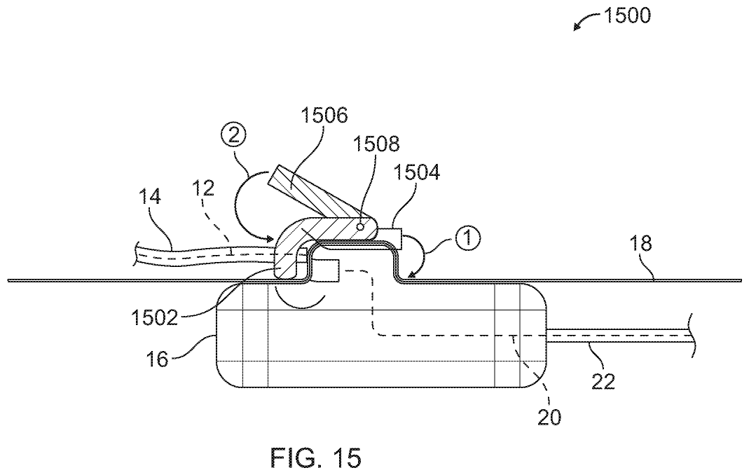

FIG. 15 illustrates a fourth connection-establishing device 1500 in accordance with some embodiments.

As shown, the connection-establishing device 1500 includes a cap 1502, bail wire 1504 coupled to the cap 15002, and an extension arm 1506 coupled to the cap 1502. The cap 1502 is configured to sit over the protruding portion 26 of, for example, the multiple-use medical device set forth above with the same procedural barrier between the connection-establishing device 1500 and the multiple-use medical device. The bail wire 1504 is coupled to the cap 1502 by ends of the bail wire 1504 in bores of the cap 1502. The bail wire 1504 is configured to secure the cap 1502 over the protruding portion 26 of the medical device by way of a hinge mechanism. The extension arm 1506 is coupled by a hinge pin 1508 to the cap 1502. The extension arm 1506 is configured to push a male connector (e.g., the male connector 202 or 302) of a single-use medical device (e.g., the PICC 10) through the procedural barrier and into a female connector (e.g., the female connector 204 or 304) of the multiple-use medical device by articulating the extension arm 1506. The extension arm 1506 is thusly configured to establish one or more functional connections (e.g., an optical connection, an electrical connection, or an optical connection and an electrical connection) across the procedural barrier between the single-use medical device on a clinician-facing side of the procedural barrier and the multiple-use medical device on a patient-facing side of the procedural barrier.

Advantageously, the connection-establishing device 1500 utilizes a mechanical advantage to decrease the force required to make the one-or-more functional connections through the procedural barrier.

### Methods

Methods include methods of using the procedural barrier-breaching connectors and the connection-establishing devices set forth above, as well as the systems thereof.

Adverting to FIG. 2, which illustrates the first connection system 200 in accordance with some embodiments, a method of the connection system 200 includes a connector-placing step, a connector-turning step, and optical connection-establishing step.

The connector-placing step includes placing a bottom portion of the base 206 of the male connector 202 into a top portion of the receptacle 212 of the female connector 204 with a procedural barrier (e.g., the sterile drape 18) therebetween.

The connector-turning step includes turning the male connector 204 by hand along a longitudinal axis thereof by way of the opposing wings 210 about a portion of the base 206 opposite the bottom portion of the base 206 while placing the male connector 202 against the procedural drape. The connector-turning step also includes cutting through the procedural barrier with the external threads 208 around the base 206 of the male connector 204 having the cutting edge.

The optical connection-establishing step includes establishing an optical connection between an optical fiber (e.g., the optical fiber of the optical-fiber stylet 12) disposed in the terminal opening in the bottom portion of the male connector 202 and another optical fiber (e.g., the optical fiber 20) disposed in the internal opening of the female connector 204 respectively held in the male connector 202 and the female connector 204.

The method can further include an electrical connection-establishing step. The electrical connection-establishing step includes establishing an electrical connection between a conductive wire of the male connector 202 and another conductive wire of the female connector 204. The conductive wire and the other conductive wire are respectively connected to the external threads 208 of the male connector 202 and the complementary internal threads 214 around the receptacle 212 of the female connector 204.

Adverting to FIGS. 3 and 4, which illustrate different views of the second connection system 300 in accordance with some embodiments, a method of the connection system 300 includes a connector-placing step, a connector-pushing step, and an optical connection-establishing step.

The connector-placing step includes placing the two-or-more prongs 306 of the male connector 302 against a procedural barrier (e.g., the sterile drape 18).

The connector-pushing step includes pushing the two-or-more prongs 306 through the procedural barrier into the two-or-more prong receptacles 310 of the female connector 304 corresponding in number to the two-or-more prongs 306 of the male connector 302. The connector-pushing step also includes cutting through the procedural barrier with the cutting edge of each prong of the two-or-more prongs 306 of the male connector 302.

The optical connection-establishing step includes establishing an optical connection between an optical fiber (e.g., the optical fiber of the optical-fiber stylet 12) disposed in the optical-fiber holder 308 along the longitudinal axis of the male connector 302 between the two-or-more prongs 306 and another optical fiber (e.g., the optical fiber 20) disposed in the optical-fiber receptacle 312 along the longitudinal axis of the female connector 304 between the two-or-more prong receptacles 310.

The method can further include an electrical connection-establishing step. The electrical connection-establishing step includes establishing an electrical connection between a conductive wire coupled to the two-or-more prongs 306 of the male connector 302 and another conductive wire coupled to the two-or-more contacts respectively disposed within the two-or-more prong receptacles 310 of the female connector 304.

Again, FIGS. 5 and 6 illustrate different views of the third connection system 500 in accordance with some embodiments; FIG. 7 illustrates the male connector 502 of the third connection system 500 in accordance with some embodiments; and FIG. 8 illustrates a breach in a procedural barrier when breached by the male connector 502 of FIG. 7 in accordance with some embodiments. A method of the connection system 500 includes a connector-placing step, a connector-turning step, and a cable-sliding step.

The connector-placing step includes placing a bottom portion of the sleeve 506 of the male connector 502 over a top portion of the optical-fiber receptacle 514 of the female connector 504 with a procedural barrier therebetween (e.g., the sterile drape 18).

The connector-turning step includes turning the male connector 502 by hand along the longitudinal axis of the sleeve 506 while placing the male connector 502 against the procedural drape. The connector-turning step also includes cutting through the procedural barrier with the cutting edge 508 around the major opening of the sleeve 506 opposite the through hole. As set forth above, the sleeve 506 of the male connector 502 has the internal threads 516 around the inside of the sleeve 506 and the female connector 504 has the complementary external threads 518 around the outside of the receptacle 514. As such, the connector-turning step can also include screwing together the male connector 502 and the female connector 504 subsequent to cutting the procedural drape in the foregoing sub-steps of the connector-turning step.

The cable-sliding step includes sliding a cable (e.g., the extension tube 14) including an optical fiber (e.g., the optical fiber of the optical-fiber stylet 12) through the through hole 512 opposite the bottom portion of the sleeve 506 of the male connector 502 into the receptacle 514 of the female connector 504. The cable-sliding step also includes establishing an optical connection between the optical fiber and another optical fiber (e.g., the optical fiber 20) disposed in the receptacle 514 of the female connector 504.

Again, FIGS. 9 and 10 illustrate different views of the fourth connection system 900 in accordance with some embodiments; and FIG. 9 illustrates the male connector 902 of the fourth connection system 900 in accordance with some embodiments. A method of the connection system 900 includes a connector-placing step, a connector-turning step, a connector-inserting step, and a cable-sliding step.

The connector-placing step includes placing the spiral cutting edge 910 of the splittable shank 908 of the male connector 902 over the longitudinal through hole of the optical-fiber receptacle 914 of the female connector 904 with a procedural barrier (e.g., the sterile drape 18) therebetween. The connector-turning step also includes turning the male connector 902 by hand along the longitudinal axis thereof while placing the male connector 902 against the procedural drape. The connector-turning step also includes cutting through the procedural barrier with the cutting edge 910 of the splittable shank 908.

The connector-inserting step includes inserting the splittable shank 908 into the longitudinal through hole of the receptacle 914 of the female connector 904 and splitting the splittable shank 908 along its length.

The cable-sliding step includes sliding a cable (e.g., the extension tube 14) including an optical fiber (e.g., the optical fiber of the optical-fiber stylet 12) through the through hole in the head 906 of the male connector 902 opposite the splittable shank 908 into the receptacle 914 of the female connector 904. The cable-sliding step also includes establishing an optical connection between the optical fiber and another optical fiber (e.g., the optical fiber 10) disposed in the receptacle 914 of the female connector 904.

Adverting to FIGS. 12, 13, and 14, which respectively illustrate a first connection-establishing device 1200, a second connection-establishing device 1300, and a third connection-establishing device 1400 in accordance with some embodiments, a method of the connection-establishing device 1200, 1300, or 1400 includes a cap-placing step, an articulating step, and a functional connection-establishing step.

The cap-placing step includes placing the cap 1202, 1302, or 1402 over the protruding portion 26 of, for example, a multiple-use medical device (e.g., the relay module 16) with a procedural barrier (e.g., the sterile drape 18) between the connection-establishing device 1200, 1300, or 1400 and the multiple-use medical device.

The articulating step includes articulating the extension arm 1204, 1304, or 1404 coupled by the hinge pin 1206, 1306, or 1406 to the cap 1202, 1302, or 1402 to push a male connector (e.g., the male connector 202 or 302) of a single-use medical device (e.g., the PICC 10) through the procedural barrier and into a female connector (e.g., the female connector 204 or 304) of the multiple-use medical device.

The functional connection-establishing step includes establishing one or more functional connections (e.g., an optical connection, an electrical connection, or an optical connection and an electrical connection) across the procedural barrier between the single-use medical device on a clinician-facing side of the procedural barrier and a multiple-use medical device on a patient-facing side of the procedural barrier.

Adverting to FIG. 15, which illustrates the fourth connection-establishing device 1500 in accordance with some embodiments, a method of the connection-establishing device includes a cap-placing step, a securing step, an articulating step, and a functional connection-establishing step.

The cap-placing step includes placing the cap 1502 over the protruding portion 26 of, for example, a multiple-use medical device (e.g., the relay module 16) with a procedural barrier (e.g., the sterile drape 18) between the connection-establishing device 1500 and the multiple-use medical device.

The securing step includes securing the cap 1502 over the protruding portion 26 of the medical device by way of a hinge mechanism of the bail wire 1504 coupled to the cap 1502 by ends of the bail wire 1504 in bores of the cap 1502.

The articulating step includes articulating the extension arm 1506 coupled by the hinge pin 1508 to the cap 1502 to push a male connector (e.g., the male connector 202 or 302) of a single-use medical device (e.g., the PICC 10) through the procedural barrier and into a female connector (e.g., the female connector 204 or 304) of the multiple-use medical device.

The functional connection-establishing step includes establishing one or more functional connections across the procedural barrier between the single-use medical device on a clinician-facing side of the procedural barrier and a multiple-use medical device on a patient-facing side of the procedural barrier.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A connection system, comprising:
a male connector including:
two or more prongs extending to a common terminal end of the male connector;
an optical-fiber holder along a longitudinal axis of the male connector between the two-or-more prongs and short of the common terminal end, the optical-fiber holder configured for holding therein a ferrule including an optical fiber; and
a female connector including:
two or more prong receptacles corresponding in number to the two-or-more prongs that extend to a common terminal end of the female connector; and
an optical-fiber receptacle along a longitudinal axis of the female connector between the two-or-more prong receptacles and commensurate with the common terminal end configured for holding therein another ferrule including another optical fiber, the male and female connectors
configured to establish an optical connection between the optical fiber of the male connector and the other optical fiber of the female connector when the optical fiber and the other optical fiber are respectively held in the male connector and the female connector.

2. The connection system according to claim 1, wherein the two-or-more prongs of the male connector and two or more contacts respectively disposed within the two-or-more prong receptacles of the female connector are electrically conductive, the male and female connectors configured to establish an electrical connection between a conductive wire of the male connector and another conductive wire of the female connector when the conductive wire and the other conductive wire are respectively coupled to the two-or-more prongs in the male connector and the two-or-more contacts in female connector.

3. The connection system according to claim 1, wherein each prong of the two-or-more prongs of the male connector include a cutting edge configured to cut through a procedural barrier when the male connector is placed against the procedural barrier and pushed by hand therethrough.

4. A connection system, comprising:
a male connector including:
a sleeve having a through hole configured to allow a cable including an optical fiber to be slidably disposed therein;
a cutting edge around a major opening of the sleeve opposite the through hole, the cutting edge configured to cut through a procedural barrier when the sleeve is placed against the procedural barrier and turned by hand along a longitudinal axis of the sleeve; and
a holed stopper configured to be disposed over the cable or a ferrule around the optical fiber, the holed stopper configured to stop the cable from slipping though the through hole; and
a female connector including:
an optical-fiber receptacle including a longitudinal through hole having an internal opening configured for holding therein another ferrule including another optical fiber, the male and female connectors configured to establish an optical connection between the optical fiber of the male connector and the other optical fiber of the female connector when the optical fiber and the other optical fiber are respectively held in the male connector and the female connector.

5. The connection system according to claim 4, wherein the sleeve of the male connector has internal threads around an inside of the sleeve and the female connector has complementary external threads around an outside of the optical-fiber receptacle for screwing together the male connector and the female connector.

6. A connection system, comprising:
a male connector including:
a head having a through hole configured to allow a cable including an optical fiber to be slidably disposed therein;
a splittable shank extending from the head configured to split along its length to allow a ferrule including the optical fiber of the cable to be pushed past a terminal end of the male connector; and
a spiral cutting edge around the splittable shank configured to cut through a procedural barrier when the male connector is placed against the procedural barrier and turned by hand along the longitudinal axis thereof; and
a female connector including:
an optical-fiber receptacle including a longitudinal through hole having an internal opening configured for holding therein another ferrule including another optical fiber, the male and female connectors configured to establish an optical connection between the optical fiber of the male connector and the other optical fiber of the female connector when the optical fiber and the other optical fiber are respectively held in the male connector and the female connector.

7. The connection system according to claim 6, wherein the splittable shank is configured to split in the optical-fiber receptacle to allow the ferrule including the optical fiber of the cable to be pushed past the terminal end of the male connector.

8. A connection-establishing device, comprising:
a cap configured to sit over a protruding portion of a first medical device with a procedural barrier between the connection-establishing device and the first medical device; and
an extension arm coupled by a hinge pin to the cap configured to push a male connector of a second medical device through the procedural barrier and into a female connector of the first medical device by articulating the extension arm and establish one or more functional connections across the procedural barrier between the second medical device on a clinician-facing side of the procedural barrier and the first medical device on a patient-facing side of the procedural barrier.

9. The connection-establishing device according to claim 8, further comprising a bail wire coupled to the cap by ends of the bail wire in bores of the cap, the bail wire configured to secure the cap over the protruding portion of the first medical device by way of a hinge mechanism.
